(19) United States
(12) Patent Application Publication
(10) Pub. No.: US 2026/0090904 A1
(43) Pub. Date: Apr. 2, 2026

(54) EXTERNAL FECAL MANAGEMENT SYSTEM AND RELATED METHODS

(71) Applicant: Carly Googe, Kathleen, GA (US)

(72) Inventor: Carly Googe, Kathleen, GA (US)

(21) Appl. No.: 19/347,495

(22) Filed: Oct. 1, 2025

Related U.S. Application Data
(60) Provisional application No. 63/701,764, filed on Oct. 1, 2024.

(51) Int. Cl. A61F 5/442; A61F 5/44

(52) U.S. Cl. CPC A61F 5/442; A61F 5/4408

(57) ABSTRACT

An improved external fecal management system comprises a wearable guardian vessel device having a border cushion that wraps around a perimeter of the wearable guardian vessel device and defines a reservoir well portion at a center of the wearable guardian vessel device; an interior tube positioned in the reservoir well portion that is coupled to a suctioning port that extends from the interior of the wearable guardian vessel device to an exterior of the wearable guardian vessel device, wherein the interior tube includes a central opening for receiving fecal material from a wearer, wherein the interior tube further includes a plurality of fenestration openings that surround the central opening of the interior tube, and a mesh layer that that is positioned within the reservoir well portion and extends over the interior tube.

### CROSS-REFERENCE TO RELATED APPLICATION

This application claims priority to co-pending U.S. provisional application entitled, “External Fecal Management Systems and Related Methods,” having application No. 63/701,764, filed Oct. 1, 2024, which is entirely incorporated herein by reference.

### BACKGROUND

Current fecal management system (FMS) are invasive and have multiple risks with many complications. Typical external fecal management systems include one or a combination of absorbent pads, diapers, and collection bags or pouches. Users of such devices are subject to the risk of skin irritations or breakdowns, including pressure ulcers, that result from the lack of a skin barrier to protect from moisture and bacteria in pressured areas of the patient skins. Internal fecal management systems including indwelling catheters, which are prone to damaging the sphincter muscles of a patient along with hemorrhaging.

There is a need for a non-invasive fecal management system that is safe for the patient and accommodating to the duties of the health care provider. Accordingly, an emergency room (ER) nurse is charged with assisting with many different critical need patients, and thus may be unable to actively assist with all of the activities of daily living (ADL's) of patients with bowel incontinence that are at risk for skin breakdown. This need exists in all types of patient care settings, including long term care (LTC), skilled nursing facilities, nursing homes, inpatient rehabilitation settings, emergency rooms, intensive/critical care units, medical-surgical units, hospice/end-of-life/palliative care units, etc.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1-3 show varying views of an exemplary embodiment of a wearable guardian vessel in accordance with the present disclosure.

FIGS. 4-6 show varying views of hand-sketch drawings of an exemplary embodiment of a wearable guardian vessel in accordance with the present disclosure.

### DETAILED DESCRIPTION

The present disclosure describes various embodiments of an external fecal management system and related methods. An exemplary external fecal management system works to provide a wearable guardian vessel comprising a contoured pad with a reservoir well for collecting fecal matter and moisture that keeps the fecal matter and moisture away from a skin of a patient (such as one that may be immobile and disposed in a hospital bed or gurney), thus providing a skin barrier, while also have a suctioning port for removal of the fecal material. Within the well of the wearable guardian vessel, a suction tube is provided with an opening for moving the fecal matter and moisture to a collection container. Unlike prior external fecal management systems, a fecal management system of the present disclosure does not utilize adhesives to attach the wearable guardian vessel to the skin of the wearer, given that adhesives can damage the skin of the patient and can lead to sores and ulcers or other injuries.

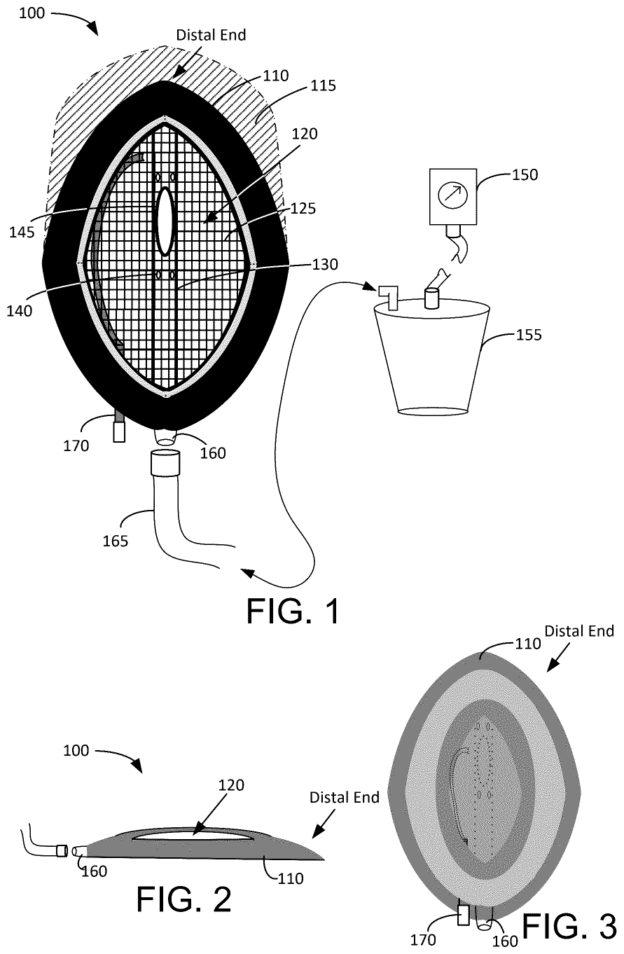

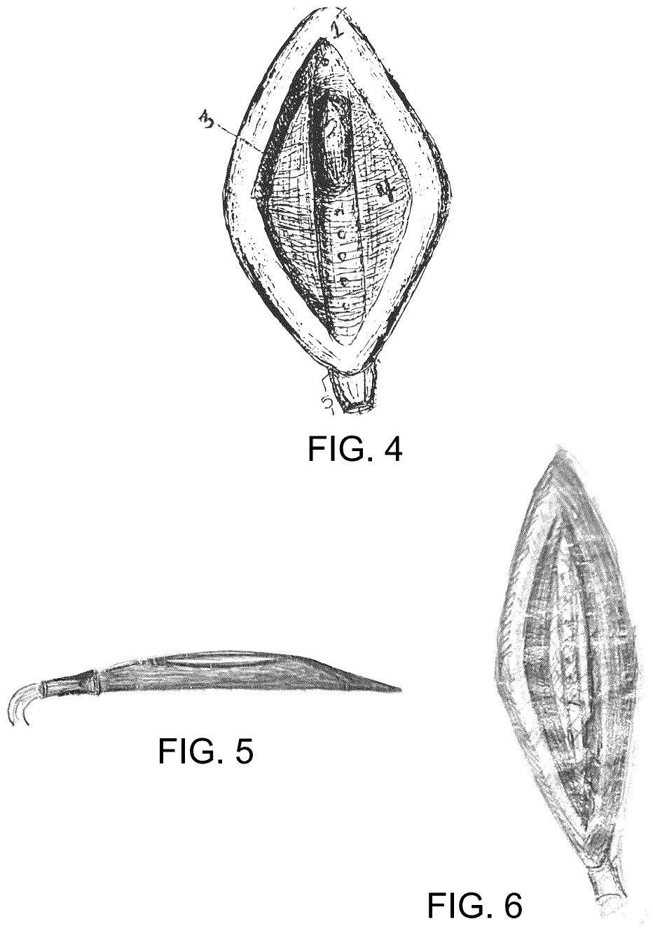

Referring now to FIG. 1, a top view of an embodiment of a wearable guardian vessel 100 is depicted. Correspondingly, FIG. 2 shows a side view and FIG. 3 shows a bottom view of an embodiment of the wearable guardian vessel 100. In various embodiments, the overall shape of the vessel 100 is curved and contoured to fit the anatomical structure of the human body. Correspondingly, FIGS. 4-6 show varying views of hand-sketch drawings of an exemplary embodiment of a wearable guardian vessel that correspond to FIGS. 1-3.

As shown in the figures, the wearable guardian vessel 100 includes an outer border or periphery 110 of waterproof foam or silicone material that gently applies to a wearer's skin without the use of an external adhesive. The border 110 defines a reservoir well 120 to prevent moisture sitting on the skin of the patient. In various embodiments, the border 110 is, but not limited to only being, 0.25 cm to 2 cm in depth and approximately 1.5 cm to 3.5 cm in width. In various embodiments, the border 110 has a beveled or graduated sloped interior surface that provides a contoured shape to a well portion of the wearable guardian vessel 100 that is designed to provide a channel for collecting fecal material and moisture from the patient (wearer). Additionally, in various embodiments, a tacky border material 115 can also be provided on a distal end of the wearable guardian vessel 100 such that the tacky border material can provide additional support for a wearer, as shown in FIG. 1. Alternatively, certain embodiments of the present disclosure may not include the tacky border material 115 and therefore, the border material 115 is not represented in FIGS. 2-6.

In various embodiments, the tacky border material 115 may extend up to 2 cm beyond a perimeter of the border 110 of the guardian vessel 100. In various embodiments, the tacky border material 115 can be a non-graduated silicone foam material, such as Mepilex® border foam material, that can gently adhere to a wearer's skin and be easily removed without sticking to the wearer's skin.

Accordingly, when the guardian vessel 100 is positioned under a patient that is lying on a bed, the only portion of the device that is in contact with a skin of the patient is the border material 110, 115 of the device and not the well portion 120 that is used to collect fecal material and moisture. In various embodiments, the reservoir well portion 120 is, but not limited to only being, approximately 14 cm in length, approximately 8 cm in width, and approximately 2 cm in depth. In various embodiments, an external surface of the well portion 120 can be composed of waterproof foam or silicone material to provide a cushion, structure, and integrity to the well portion and to prevent deformation from pressure and weight from a wearer and the underlying bed, while also providing a comfortable fit.

As discussed, the interior surface of the guardian vessel 100 tapers down from the border material 110 to the reservoir well portion 120, where the well portion 120 provides a channel that holds an internal tube 130 having one large central drain 140 opening and several fenestration openings 145 (e.g., 2 openings spaced bilaterally above the central drain opening and 2 openings spaced bilaterally below the central drain opening) running along the center axis of the tube 130. In various embodiments, the fenestration openings 145 may be located on each side of the internal tube 130. These openings 140, 145 provide a suctioning force (via a vacuum pump 150) that draws or pulls fecal matter and moisture into the reservoir well 120 and through the central drain opening 140 of the internal tube 130 that is connected to a collection container 155 for removal of the fecal material. In various embodiments, the inner tube is, but not limited to only being, approximately 1.5 cm in diameter, 15 cm in length, with the central drain opening 140 being approximately 4 cm in length×1.25 cm in width, with the fenestration openings 145 being approximately 0.5 cm in diameter. In various embodiments, a continuous suctioning force can be applied to the inner tube 130 via the vacuum pump 150. In various embodiments, the suctioning force can be adjustable to provide periodic or intermittent suctioning forces or can be configured to provide on-demand/manual activation of suctioning actions via the vacuum pump 150. Additionally, in various embodiments, the size of the central drain opening 140 may be greater than 4 cm in length and/or 1.25 cm in width (e.g., 8 cm in length×1.25 cm in width) to accommodate larger patients.

In various embodiments, the inner tube 130 extends to a suctioning port 160 for coupling to suction tubing 165, such as a portable or wall vacuum unit 150 that is found in many patient care areas. The suctioning port 160 may couple to the suction tubing 165 via a snap fit, interference fit, friction fit, screwed, clipped on, or by way of various temporary adhesion and securing mechanism, such as but not limited to a luer lock. In various embodiments, the suctioning port 160 is, but not limited to only being, approximately from 0.75 cm to 1.5 cm in diameter. A collection container 155 can be fluidly coupled to the suction tubing 165 to receive the fecal material. The collection container 155 may be comprised of a translucent material and a graduated scale may be printed on the collection container 155 for determining the fill level of the collection container 155. A negative pressure or suctioning action may be generated by the vacuum pump 150 that is also coupled to the suction tubing 165 in order to draw the fecal material from the wearable guardian vessel 100 to the collection container 155.

In various embodiments, the reservoir well portion 120 of the wearable guardian vessel 100 includes a mesh layer 125 that overlaps the channel and internal tube components, where, in various embodiments, the mesh layer 125 provides rectangular or circular/oval holes that allow for fecal particles of a smaller size than the holes to pass through. In various embodiments, holes of the mesh layer 125 are, but not limited to only being, approximately 0.25 cm in diameter or 0.25 cm×0.25 cm in area. Thus, particles larger than the hole size may be available for evaluation by a medical attendant, nurse, or doctor, such as blood clots due to gastrointestinal hemorrhaging, or undigested food. In various embodiments, the mesh layer may be composed of a silicone material that provides a layer of protection from the suctioning action of the inner tube 130 contacting a skin of the patient (wearer).

In various embodiments, in addition to the suction tubing 165, an irrigation tubing (not shown) can also be coupled to the wearable guardian vessel 100. Accordingly, in various embodiments, the wearable guardian vessel 100 features an irrigation connector 170 port, where the irrigation connector port 170 is coupled to or comprises a tube that travels to an interior of the well portion 120 of the wearable guardian vessel 100 to the left or right of the interior tube 130. In various embodiments, the irrigation tubing is, but not limited to only being, approximately 2 mm in diameter and approximately 20 cm in length, where the irrigation connector port 170 is compatible for coupling with syringes so that the syringes can push or flush a stream of liquid (e.g., saline, water, etc.) into the well portion 120 of the vessel 100, which can aid in loosening or liquefying the stool during its removal through the inner channel. Accordingly, in various embodiments, the irrigation connector port 170 features a luer lock connector that is compatible with luer lock syringes and/or IV tubing (e.g., 20 CC tubing). Thus, in various embodiments, a saline bag can be coupled to the irrigation connector port 170 and can be configured to continuously or intermittently flush or drip saline into the inner channel of the wearable guardian vessel 100.

During use, the wearable guardian vessel 100 can be positioned over the anus and sacral area of a patient with the suctioning port 160 positioned near a groin area and/or between the patient's legs (in a direction towards a foot of the patient's bed). The distal end of the vessel 100 covers the perineum region of the patient (wearer) and extends to cover a portion of the patient's groin region, such as the penis root or labia). Since the human anatomy is very diverse between men and women and among individuals of the same sex, different size options may be provided for the guardian vessel in accordance with various embodiments of the present disclosure. Accordingly, suction tubing 165 can be coupled to the suctioning port 160 and a vacuum pump 150 can be activated to provide continuous or intermittent suction flow from 20-60 mmHg that is capable of removing fecal matter from the wearable guardian vessel 100 to a collection container 155.

In general, patients that are bedridden in a hospital setting are treated with a skin barrier cream on the patient's sacrum region. Accordingly, without the need of an adhesive aid, the foam or silicone barrier material 110, 115 of the wearable guardian vessel 100 will sufficiently stick to the cream and/or skin of the patient such that it will not move out of place as the patient is positioned on top of the vessel as the patient lies on their back in the hospital bed. By not utilizing an adhesive to apply the wearable guardian vessel 100 to a patient, skin cells are not pulled away from patient thereby weaking the skin barrier that is necessary to avoid potential skin injuries. Alternatively, in certain embodiments, a backside of the wearable guardian vessel 100 may have adhesive strips to secure the pad to a cloth of an undergarment, such as an incontinence brief that is worn by the patient, or may have some other form of fastener, such as a hook and loop material or a tacky material that clings to a fabric of the undergarment but would not pull away skin cells of a patient when the wearable guardian vessel 100 is removed.

The exemplary wearable guardian vessel 100 of the present disclosure satisfies the need for a non-invasive fecal management system that is safe for the patient and accommodating to the duties of the health care provider. Accordingly, such a device can be used in various types of patient care settings, including long term care (LTC), skilled nursing facilities, nursing homes, inpatient rehabilitation settings, emergency rooms, intensive/critical care units, medical-surgical units, hospice/end-of-life/palliative care units, etc. Benefits of its use include improved hemodynamic monitoring with accurate intake and output assessment, safely securing fecal matter in a collection container via a vacuum pump, thereby decreasing the risk of cross-contamination, decreasing the likelihood of skin breakdown and improving the healing of sacral pressure wounds, improving skin barrier protection by keeping the patient's skin more dry, being non-invasive, not causing rectal pressure wounds, not causing rectal fissures, not contributing towards gastrointestinal bleeding, increasing patient comfort, decreasing hospital acquired pressure injuries with at risk patients and associated costs to facility, not requiring a medical doctor order to administer as the treatment is non-invasive, optionally providing continuous, intermittent, or on-demand irrigation with fluid if needed for soft/non-liquid stool, etc. Moreover, the wearable guardian vessel protects the skin barrier at pressured areas of the patient's skin from moisture and bacteria that are associated with fecal material.

It is also to be understood that the terminology used herein is for purposes of describing particular embodiments only, and is not intended to be limiting. It must be noted that, as used in the specification and the appended claims, the singular forms “a,” “an,” and “the” include plural referents unless the context clearly dictates otherwise.

Unless defined otherwise, all technical and scientific terms used herein have the same meanings as commonly understood by one of skill in the art to which the disclosed invention belongs. Publications cited herein and the materials for which they a

It is also to be understood that the specific devices and processes illustrated in the attached drawings, and described in the foregoing specification, are exemplary embodiments of inventive concepts defined in the present application. Hence, specific dimensions and other physical characteristics relating to the embodiments disclosed herein are not to be considered as limiting, unless the following claims expressly state otherwise.

A number of materials are identified as suitable for various aspects of the present disclosure. These materials are to be treated as exemplary and are not intended to limit the scope of the claims. Although any methods and materials similar or equivalent to those described herein can also be used in the practice or testing of the present disclosure, a limited number of exemplary methods, devices, and materials are described herein.

Those skilled in the art will recognize, or be able to ascertain using no more than routine experimentation, many equivalents to the specific embodiments of the present disclosure described herein. Such equivalents are intended to be encompassed by the following claims.

It should be emphasized that the above-described embodiments are merely possible examples of implementations, merely set forth for a clear understanding of the principles of the present disclosure. Many variations and modifications may be made to the above-described embodiment(s) without departing substantially from the principles of the present disclosure. All such modifications and variations are intended to be included herein within the scope of this disclosure.

Therefore, at least the following is claimed:

1. An external fecal management system comprising:
a wearable guardian vessel device having a border cushion that wraps around a perimeter of the wearable guardian vessel device and defines a reservoir well portion at a center of the wearable guardian vessel device;
an interior tube positioned in the reservoir well portion that is coupled to a suctioning port that extends from the interior of the wearable guardian vessel device to an exterior of the wearable guardian vessel device, wherein the interior tube includes a central opening for receiving fecal material from a wearer, wherein the interior tube further includes a plurality of fenestration openings that surround the central opening of the interior tube, wherein the central opening and the fenestration openings are in fluidic communication with the suctioning port; and
a mesh layer that that is positioned within the reservoir well portion and extends over the interior tube;
wherein the wearable guardian vessel device is sized to cover an anus of a wearer and extend over a portion of a groin region of the wearer when the wearer is situated over the wearable guardian vessel device.

2. The system of claim 1, further comprising a vacuum pump that is coupled to the suctioning port via suction tubing, wherein the vacuum pump is configured to provide a suctioning force to the interior tube.

3. The system of claim 1, wherein the wearable guardian vessel device further comprises an irrigation tubing positioned within the reservoir well portion that is coupled to an irrigation port that extends from the interior of the wearable guardian vessel device to the exterior of the wearable guardian vessel device, wherein the irrigation tubing is in fluidic communication with the irrigation port.

4. The system of claim 1, wherein the border cushion is comprised of foam material or silicone material that is waterproof.

5. The system of claim 1, wherein the mesh layer is comprised of silicone material.

6. The system of claim 1, wherein an external backside of the wearable guardian vessel device is comprised of a foam material or a silicone material that is waterproof.
